(19) United States
(12) Patent Application Publication
(10) Pub. No.: US 2021/0128822 A1
(43) Pub. Date: May 6, 2021

(54) IMPROVED INTRAVENOUS INFUSION SET

(71) Applicant: POLY MEDICURE LIMITED, Faridabad-Haryana (IN)

(72) Inventor: Rishi Baid, New Delhi (IN)

(73) Assignee: POLY MEDICURE LIMITED, Faridabad-Haryana (IN)

(21) Appl. No.: 17/053,019

(22) PCT Filed: May 3, 2019
(86) PCT No.: PCT/IB2019/053641

§ 371 Date: Nov. 4, 2020

(30) Foreign Application Priority Data
May 4, 2018 (IN) 201811017011

(51) Int. Cl. A61M 5/14; A61M 5/38; A61M 39/28; A61M 39/10

(52) U.S. Cl. CPC A61M 5/1411; A61M 5/38; A61M 2005/1657; A61M 39/10; A61M 2039/1077; A61M 39/285

(57) ABSTRACT

An improved intravenous infusion set (10) to administer delivery of intravenous fluid to a patient, said intravenous infusion set (10) comprising: a drip chamber (12), a flexible infusion line (30) of sufficient length for fluid delivery to the patient and connected the drip chamber (12); a roller clamp (34) arranged movably along the length of the flexible infusion line (30), an air vent (18) with a cap arranged at the distal end (54) of the drip chamber (12) for management of air in the infusion system, said drip chamber (12) has a spike (14) on a distal end (54) and an infusion fluid outlet (20) at a proximal end (52) of the drip chamber (12), a fluid flow regulating member (36) sealed within the drip chamber (12) over said infusion fluid outlet (20) and wherein said infusion fluid outlet (20) configured to house a flow path adapter (22) which facilitates in administering continuous air free delivery of intravenous fluid to patients under gravitational pull.

### FIELD OF THE INVENTION

The present invention relates to an intravenous infusion set providing delivery of intravenous fluid to patients. More particularly, the invention relates to an improved intravenous infusion set with a drip chamber having a fluid flow regulating member to administer continuous air free delivery of intravenous fluid to patients under gravitational pull. The invention also relates to a flow path adapter arranged in the outlet of drip chamber which facilitates in administering continuous air free delivery of intravenous fluid to patients under gravitational pull.

### BACKGROUND OF THE INVENTION

Intravenous infusion sets with a drip chamber having fluid flow regulating members are already known and used to administer controlled quantities of intravenous fluid (for example glucose solution, saline solution, medicine, blood, blood components, etc.) into a patient's body seeking treatment. Such infusion sets are either pump driven or gravity driven infusion devices.

Gravity driven infusion devices typically include a drip chamber with or without a fluid flow regulating member, a length of flexible infusion line to connect the lower end of the drip chamber to a standard connector. The connector is then connected via conventional access means such as a needle, a catheter or the like to a patient. The distal end of the drip chamber is provided with a spike which is used to pierce a fluid source, such as a bag or bottle containing a specified infusion fluid.

Generally, the whole assembly comprising the fluid source, the spike and the drip chamber is hung on a hanger to provide sufficient height so that the infusion fluid is driven by gravitational force allowing the infusion fluid to flow downward towards the patient from the fluid source. A standard fluid flow adjustment device, for example a roller clamp, is provided on the flexible infusion line between the drip chamber and the connector for regulating the rate of flow of the infusion fluid. The roller clamp can be used to stop the flow of fluid through the flexible infusion line as soon as the fluid source containing the fluid is near to being empty or is empty.

However, various problems and disadvantages are associated with such infusion devices comprising a drip chamber with or without a fluid flow regulating member. One of the major problems commonly faced is that a certain amount of air in the form of bubbles may get into the blood circulation of the patient accidently during the infusion procedure, which may lead to air embolism. The air embolism in a patient may cause breathlessness, chest pain, stroke, wheezing, fast heartbeat, loss of consciousness or the like, and may lead to death.

Further, an air bubble present in the drip chamber can spread over the surface of the fluid flow regulating member for example, the hydrophilic filter and thereby effectively cut off further fluid flow. In some instances, due to pressure, which may be due to air as well as fluid, the flow path may get obstructed or collapse which may cutoff further fluid flow.

Accordingly, it is desired to provide an improved intravenous infusion set having a drip chamber with a fluid flow regulating member and other means to administer continuous air free delivery of intravenous fluid to patients, which overcomes the above-discussed disadvantages and is inexpensive to manufacture, efficient, effective and simple in its construction and use.

These and other aspects of the present invention will be better understood in the description below.

### SUMMARY AND OBJECTS OF THE INVENTION

A primary object and advantage of the present invention is to provide an improved intravenous infusion set to administer continuous air free delivery of intravenous fluid to patients.

Another object and advantage of the present invention is to provide an improved intravenous infusion set to achieve auto-stop function once the drip chamber is empty.

Another object and advantage of the present invention is to provide an improved intravenous infusion set which is inexpensive to manufacture, efficient, effective and simple in its construction and use.

Another object and advantage of the present invention is to provide an improved intravenous infusion set which prevents the instances of air embolism.

Another object and advantage of the present invention is to provide an improved intravenous infusion set with a flow path adapter arranged in the outlet of drip chamber which facilitates in administering continuous air free delivery of intravenous fluid to patients under gravitational pull.

Another object and advantage of the present invention is to provide an improved intravenous infusion set having a drip chamber with a fluid flow regulating member which prevents the air from entering the flexible infusion line through the drip chamber as soon as it comes into contact with air.

Another object and advantage of the present invention is to provide an improved intravenous infusion set having a drip chamber with a fluid flow regulating member which prevents infusion related infection.

Another object and advantage of the present invention is to provide an improved intravenous infusion set which prevents contaminants, microorganism, infectious agents or the like to enter the flexible infusion line.

Another object and advantage of the present invention is to provide an improved intravenous infusion set which helps reducing work load with respect to its installation and use, and ensures safe priming which may be crucial in critical conditions.

Another object and advantage of the present inventio is to provide a drip chamber for use in the intravenous administration set which is both air blocking and air eliminating whereby air contained in the drip chamber can be eliminated from the system without disassembly.

Yet another object of the invention is to provide a fluid delivery system wherein the fluid flow regulating member remains wetted once infused by the contents of a fluid container, thereby blocking the passage of air and also permitting easy and safe restart of fluid flow from another filled container without the need for re-priming the system.

Another object and advantage of the present invention is to provide an improved intravenous infusion set, which can help in preparing a series of infusions at once, and which is quicker and more hygienic than ever before.

Accordingly, the present invention relates to an improved intravenous infusion set to administer continuous air free delivery of intravenous fluid to a patient, said intravenous infusion set comprising: a drip chamber, a flexible infusion line of sufficient length connecting a proximal end of the drip chamber to a standard connector at the patient end so that a needle or a catheter could be connected to the patient; a roller clamp arranged between the drip chamber and the standard connector being movable along the length of the flexible infusion line, said drip chamber has a spike on the distal end and an infusion fluid outlet at the proximal end of the drip chamber, said infusion fluid outlet is covered with a fluid flow regulating member and provided with a flow path adapter which facilitates in administering continuous air free delivery of intravenous fluid to patients under gravitational pull. The flow path adapter is made of a suitable chemically inert plastic material and is preferably rigid. Alternatively, the flow adapter can be made of a flexible plastic material as well.

The distal end of the drip chamber is also provided with an air vent with a cap. The air vent has a filter and used for management of air in the infusion system.

The fluid flow regulating member of the invention is a hydrophilic membrane, which is permeable to fluid and impermeable to air. The hydrophilic membrane has a circular shape. However, the hydrophilic membrane can have other geometrical shapes such as oval, semi-circular etc. Preferably the material of hydrophilic membrane can be flexible and can be sheet or film, tube or fiber, or plug form. The material can be a fabric, such as a nonwoven, woven, or knit fabric, or a scrim. The material can be made of paper such as filter paper, or a cloth, or a metal mesh. It can also be made of fiberglass, cellulosic, ceramic or the like. The material can also be a porous polymeric film or membrane, synthetic or natural, where the pores form the interstices or passageways. Representative polymers useful in the material include polyamide, nylon, polyurethane, polyester, polycarbonate, polyvinylidene fluoride, polyacrylic, polyolefins such as polyethylene and polypropylene, polytetrafluoroethylene, polyvinyl chloride and the like.

Said fluid flow regulating member, which as stated is a hydrophilic membrane, usually sealed over the infusion fluid outlet at the proximal end of the drip chamber. The fluid flow regulating member is arranged in the drip chamber in a close fit manner eliminating the possibility of air being trapped or air bubbles being sucked into the infusion line. The fluid flow regulating member has a proximal face and a distal face. The proximal face of the fluid flow regulating member includes a sealing area which is used for sealing the membrane. The sealed fluid flow regulating member covers the outlet so that air bubbles and contaminants are prevented from entering the body of the patient during an infusion therapy. The said fluid flow regulating member can also be arranged by different means and methods over the infusion fluid outlet at the proximal end of the drip chamber, for example, the fluid flow regulating member can be arranged over a floating disc. The fluid flow regulating member is effective when wet. In one embodiment, the fluid flow regulating member, i.e. the hydrophilic membrane, can be sealed on the proximal face of the disc. Alternatively, the fluid flow regulating member, i.e. the hydrophilic membrane, can be sealed on the distal face of the disc. Further alternatively, the hydrophilic membrane can be sealed on both the proximal face and distal face of the disc.

The air bubbles are prevented from entering the infusion tubing through the drip chamber of an infusion set due to the hydrophilic membrane which also eliminates the possibility of air trapping. The hydrophilic membrane has a defined wetting time and a defined mean pore size that provides a defined bubble pressure point under gravitational pull to permit continuous air free infusion liquid flow there-through. The invention further provides an intravenous infusion set, wherein air bubbles cannot be sucked into the infusion line due to any deformation of the drip chamber caused by priming, or through the hydrophilic membrane due to the adjustment of the roller clamp along the infusion tubing, thereby ensuring safe replacement of the infusion bottle during multiple infusion procedures, thus preventing the occurrence of air embolism in patients, especially when the drip chamber is empty.

The proximal end of drip chamber in particularly lower part thereof ends in an infusion fluid outlet which is tubular and configured to house a flow path adapter. The flow path adapter has an upper portion and a lower portion and a through hole therethrough for fluid flow. The lower portion of the flow path adapter is connected to a flexible infusion line in a leak proof arrangement. The upper portion of the flow path adapter is received in the outlet in a leak proof arrangement. The presence of flow path adapter removes the chances of any potential functional failure during infusion therapy in that the flow path never gets obstructed. The fluid flow regulating member i.e. hydrophilic membrane covers the outlet so that air bubbles and contaminants are prevented from entering the body of the patient during an infusion therapy.

In accordance with another embodiment of the present invention, the intravenous infusion set comprises a drip chamber. The drip chamber has two parts i.e. an upper part and a lower part, which are a hollow circular cylinder with a uniform wall thickness. Both the upper parts and lower parts can be flexible or rigid. Both parts are connected by a joining member in a leak proof arrangement. The flexible part of the drip chamber is used for the purposes of priming. The upper part of the drip chamber is also provided with an air vent with a cap. The air vent has a filter and used for management of air in the infusion system.

The drip chamber can have a single structure.

The proximal end of drip chamber in particularly lower part thereof ends in an infusion fluid outlet which is tubular and configured to house a flow path adapter. Said infusion fluid outlet is covered with a fluid flow regulating member. The flow path adapter is so adjusted and housed in the infusion fluid outlet of the drip chamber that the fluid flow regulating member rests over the flow path adapter impeccably once said fluid flow regulating member is sealed by the means and methods such as energy director for ultrasonic welding or the like. Consideration is taken into account to take care for any damage to the fluid flow regulating member due to interference between a distal end of flow path adapter and the fluid flow regulating member to avoid any functional failure.

The flow path adapter has an upper portion having a distal extension, a middle portion and a lower portion and a through hole therethrough for fluid flow. The lower portion of the flow path adapter is connected to a flexible infusion line in a leak proof arrangement. The flow path adapter in its upper portion in particularly in a distal extension comprises one or more slits. In one of the embodiment, the flow path adapter has at least one slit. In other embodiments, the flow path adapter has at least four slits, which are periodically placed across the distal extension 160 of the flow path adapter.

The dimension of the slits is so designed so that it only allows the flow of fluid from the slits and stop bubbles from entering into the infusion line. The opening at the top in the flow path adapter i.e. the opening in a distal extension is sealed with a knob in a leak proof arrangement. The distal extension has one or more projections and ridges/depression which work as a bubble buster and stop the flow of bubble into the infusion line.

The flow path adapter has a unique design in that it eliminates collapsing of flexible infusion line affecting flow of liquid. This unique design of the flow path adapter also eliminates the chances of trapped air or bubbles going into the infusion line. The working principle behind such elimination of the trapped air or bubbles going into the infusion line is that once the bubble or trapped air hits the upper portion having a distal extension of the flow path adapter i.e. one or more projections and ridges/depression provided therein, the bubble or trapped air breaks down. The area or passage provided is so less that any chances of trapped air or bubbles passing through one or more slits provided in the flow path adapter and into its passage and in turn into the infusion line is eliminated. Thus, the improved design of the flow path adapter deals effectively with the most common yet serious issue of travelling of the bubble or trapped air into the infusion line which is eliminated providing safety to the end user.

The present invention embodies that the structure of the drip chamber can comprise shapes and configurations such as square, oval, rectangular, triangular, combinations thereof or the like etc. either wholly or partly along the length of the drip chamber.

The present invention embodies that the structure of the fluid flow regulating member, which is permeable to fluid and impermeable to air, can comprise shapes and configurations such as square, oval, rectangular, triangular, combinations thereof or the like etc.

The drip chamber is made of a suitable chemically inert plastic material. The present invention embodies that the drip chamber can also be made of other suitable materials such as ceramic, wood, metals and combinations thereof etc. Preferably, the wall of the drip chamber is transparent and flexible, being made of a flexible material. The wall of the drip chamber can also be rigid, being made of a rigid material.

The drip chamber comprises a hollow upper part and lower part, preferably formed as a hollow cylinder, further preferably as a hollow circular cylinder, and further preferably having a uniform wall thickness. The upper and lower part are connected by a joining member. The joining member may be a ring or has a structure which joins both the parts in a leak proof arrangement. This ensures that the upper and lower parts, which forms a part of the drip chamber, can be manufactured easily and cost efficiently, e.g. by extrusion.

The fluid flow regulating member is sealed on a sealing leg or ring which extends within the lower part of the drip chamber adjoining the inner wall adjacent to the infusion fluid outlet. The sealing contact area of the fluid flow regulating member with the sealing leg or ring forms a sealing area. Preferably, the sealing leg or ring is circularly arranged, while being distanced from the inner wall and/or the wall of the lower part of the drip chamber. The sealing area is arranged such that a remaining inner area under the fluid flow regulating member helps to ensure that liquid/fluid pressure inside the drip chamber results in equal forces on the inner area thereby balancing the fluid flow regulating member, so that it is less likely to bulge or deform.

A circular rim is provided below the said sealing leg or ring in the inner area. The circular rim supports the fluid flow regulating member. The circular rim may touch or may not touch when the fluid flow regulating member is sealed over the sealing ring.

The sealing ring and circular rim ensure that applying a force to the drip chamber in a direction perpendicular to the main extension direction of the lower part will not affect the fluid flow regulating member. This ensures that fluid flow regulating member maintains its shape and ensure that the exerted force is not transferred to the fluid flow regulating member. In other words, the fluid flow regulating member remains tightly sealed on the sealing ring or leg without any deformity or malfunction.

### BRIEF DESCRIPTION OF THE ACCOMPANYING DRAWINGS

The foregoing and other objects, features, and advantages of the invention will be apparent from the following detailed description taken in conjunction with the accompanying drawings, wherein:

FIG. 1 is a schematic drawing of the intravenous infusion set with a drip chamber of the present invention;

FIG. 2 is a cross-section of the intravenous infusion set with a drip chamber of the present invention;

FIG. 3A is a side view of a cross section of a lower part of the drip chamber of the present invention.

FIG. 3B is another side view of a cross section of a lower part of the drip chamber with a fluid flow regulating member and a flow path adapter arranged in the outlet of said drip chamber which facilitates in administering continuous air free delivery of intravenous fluid to patients under gravitational pull according to the present invention.

FIGS. 4A, 4B & 4C are a cross-sectional side view, a side view and a perspective view respectively of a flow path adapter according to the present invention.

FIG. 5 is an intravenous infusion set according to other embodiment of the present invention.

FIG. 6 is a cross-section of the intravenous infusion set with a drip chamber having a flow path adapter according to the embodiment of FIG. 5 of the present invention.

FIGS. 7(A)-(E), are views of the flow path adapter(s) used with the embodiment of FIG. 5 according to the present invention.

### DETAILED DESCRIPTION OF THE INVENTION

Generally speaking, the term “proximal” refers to a region of the device or parts thereof or a location on the device, which is closest to, for example, a user using the device. In contrast to this, the term “distal” refers to a region of the device, which is farthest from the user, for example, the distal region of a needle will be the region of a needle containing the needle tip which is to be inserted e.g. into a patient's vein.

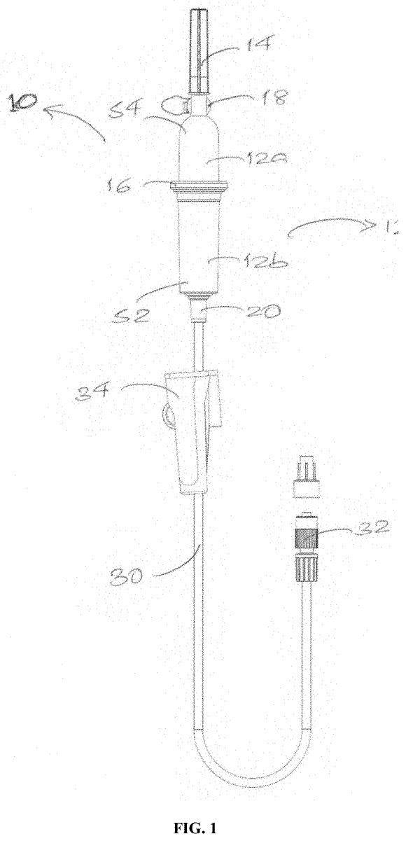

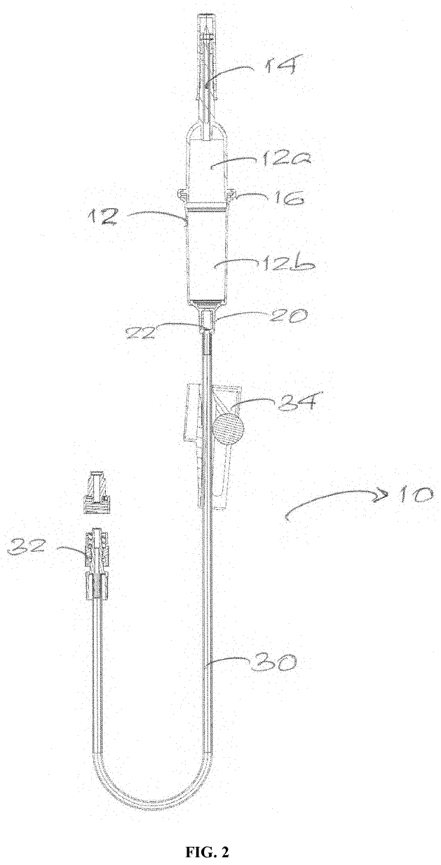

Referring to FIGS. 1 and 2, an intravenous infusion set 10 is illustrated. The intravenous infusion set 10 comprises a drip chamber 12. The drip chamber 12 has two parts i.e. an upper part 12a and a lower part 12b, which are a hollow circular cylinder with a uniform wall thickness. In one embodiment, both the upper 12a and lower 12b parts are flexible. In another embodiment, the upper part 12a is flexible whereas the lower part 12b is rigid. In yet another embodiment, the upper part 12a is rigid whereas the lower part 12b is flexible. Both of the parts 12a, 12b are connected by a joining member 16 in a leak proof arrangement. The flexible part of the drip chamber 12 is used for the purposes of priming. The upper part 12a of the drip chamber 12 is also provided with an air vent 18 with a cap. The air vent 18 has a filter and used for management of air in the infusion system.

The drip chamber 12 is provided with a spike 14 at a distal end of the upper part 12a. The spike 14 has a pointed tip which is used to pierce a fluid bag/bottle (not shown) so that the infusion fluid will be pulled by gravity into the drip chamber 12. The spike 14 is provided with slits or holes for fluid to flow into the drip chamber 12 from the fluid bag/bottle to the patient.

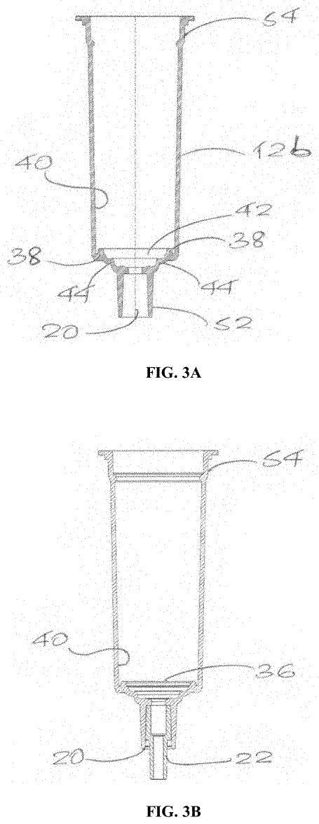

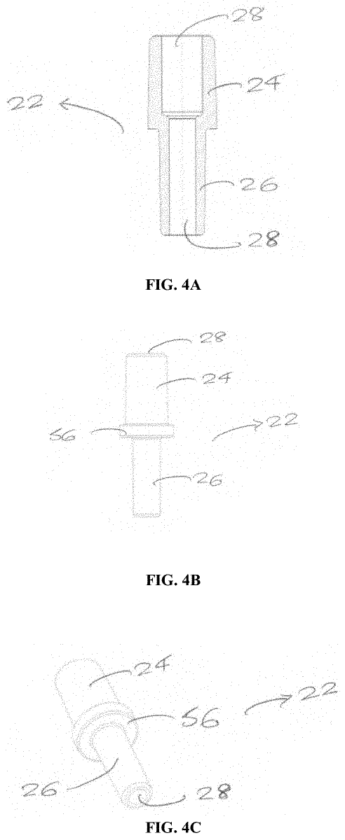

The proximal end 52 of the lower part 12b of drip chamber 12 as illustrated in FIGS. 3A and 3B ends in an infusion fluid outlet 20 which is tubular and configured to house a flow path adapter 22 as shown in FIG. 3B. The distal end 54 of lower part is connected to the proximal end 52 of the upper part by a joining member 16. The flow path adapter 22 as shown in FIGS. 4A-4C has an upper portion 24 and a lower portion 26 and a through hole 28 therethrough forming a passage for fluid flow. The upper portion 24 and a lower portion 26 are divided by a rim 56. The passage of the upper portion 24 has a bigger diameter than the passage of the lower portion 26. The lower portion 24 of the flow path adapter 22 is connected to a flexible infusion line 30 in a leak proof arrangement. The upper portion 24 of the flow path adapter 22 is received in the outlet 20 in a leak proof arrangement. One end of the flexible infusion line 30 of sufficient length as shown in FIGS. 1 and 2 connects to a standard connector 32 at the patient end and other end of the infusion line 30 connects to the lower portion 26 of the flow path adapter 22 in a leak proof arrangement. The presence of flow path adapter 22 removes the chances of any potential functional failure during infusion therapy in that the fluid flow path never gets obstructed.

The outer diameter of the upper portion 24 has a close-fit ratio with the inner diameter of infusion fluid outlet 20.

A fluid flow adjustment device, such as a roller clamp 34, is arranged being movable along the length of the flexible infusion line 30. The roller clamp 34 can be gradually adjusted so that the infusion line 30 could be squeezed proportionally to change the infusion rate and can also help in changing the fluid source, i.e. bottle or bag for multiple infusion procedures.

A clip (not shown) can also be arranged between the drip chamber 12 and the roller clamp 34 along the length of the flexible infusion line 22. The clip is configured to stop the flow of infusion fluid instantly by a clipping action.

Referring now to FIG. 3A a lower part 12b of the drip chamber 12 is illustrated, preferably formed as a hollow cylinder, further preferably as a hollow circular cylinder, and further preferably having a uniform wall thickness. The proximal end 52 of lower part 12b has a funnel shape. As shown in FIG. 3B, a fluid flow regulating member 36 is sealed on a sealing leg or ring 38 which extends within the circumference of an inner wall 40 adjacent to the infusion fluid outlet 20 in the lower part 12b of the drip chamber 12. The sealing contact area of the fluid flow regulating member 36 with the sealing leg or ring 38 forms a sealing area. Preferably, the sealing leg or ring 38 is circularly arranged, while being distanced from the inner wall 40 of the lower part 12b of the drip chamber 12. The sealing area is arranged such that a remaining inner area 42 beneath the fluid flow regulating member 36 helps to ensure that liquid/fluid pressure inside the drip chamber 12 results in equal forces on the inner area 42 thereby balancing the fluid flow regulating member 36, so that it is less likely to bulge or deform or dysfunction.

A circular rim 44 adjoining circumferentially the inner wall 42 is provided below the said sealing leg or ring 38 in the inner area 42. The circular rim 44 supports the fluid flow regulating member 36. The circular rim 44 may touch or may not touch when the fluid flow regulating member 36 is sealed over the sealing ring 38 and wetted by the fluid in operation.

The sealing ring 38 and circular rim 44 ensure that applying a force to the drip chamber 12 in a direction perpendicular to the main extension direction of the lower part will not affect the fluid flow regulating member 36. This ensures that fluid flow regulating member 36 maintains its shape and ensure that the exerted force is not transferred to the fluid flow regulating member 36. In other words, the fluid flow regulating member 36 remains tightly sealed on the sealing ring or leg 38 without any deformity or malfunction.

As shown in FIG. 3B, the sealed fluid flow regulating member 36 i.e. hydrophilic membrane covers the outlet 20 so that air bubbles and contaminants are prevented from entering the body of the patient during an infusion therapy. No air passes when the fluid flow regulating member 36 is wet. The fluid flow regulating member 36 extends perpendicular to the main extension direction of the drip chamber 12, in particular of the lower part 12. The fluid flow regulating member 36 has a proximal face 46 and a distal face 48. The proximal face 46 of the fluid flow regulating member 36 includes a sealing area. The sealing area of the fluid flow regulating member 36 is sealed onto said sealing leg or ring 38 and is arranged such that a remaining inner area 42 and a remaining outer area 50 of the fluid flow regulating member 36. The sealed fluid flow regulating member 36 covers the outlet 20 so that air bubbles and contaminants are prevented from entering the body of the patient during an infusion therapy.

Alternatively, the fluid flow regulating member 36, i.e. the hydrophilic membrane, can be sealed on the distal face 48. Further alternatively, the fluid flow regulating member 36 can be sealed on both the proximal face 46 and distal face 48.

The act of sealing referred above, for example sealing of fluid flow regulating member 36 with the drip chamber 12 in particularly with sealing leg or sealing ring 38, takes place by heat sealing, adhesive sealing, ultrasonic welding, heated die, radio frequency, mechanical seal, insert molding, laser welding, press/snap fit, annular ring with groove fitment, clamping, gluing or by the like processes etc.

The fluid flow regulating member 36 which is a hydrophilic membrane of the present invention has a strong affinity for fluid/liquid while restricting air from attaching to its surface. As a result, the membrane is permeable to fluid/liquid and impermeable to air. A fully wetted hydrophilic membrane will allow fluid/liquid to flow through while acting like a barrier to air bubbles passing across it. Under normal conditions, flow of fluid/liquid from a fully wetted membrane is expected to start under gravity pull more readily. Hence with the present membrane setup, the fluid in the drip chamber will be ready to flow once it is filled up. No milking of the roller clamp is required to initiate the flow.

In the present invention, said hydrophilic membrane will automatically stop the fluid flow once the fluid in the drip chamber 12 is empty and as long as the membrane is still totally wet. As such, no air will get into the flexible infusion line 30 during and at the end of the infusion procedure. Furthermore, because the same intravenous infusion set 10 can be used for multiple infusions, the material cost and clinical waste is greatly reduced. This is one of the advantages of the present invention.

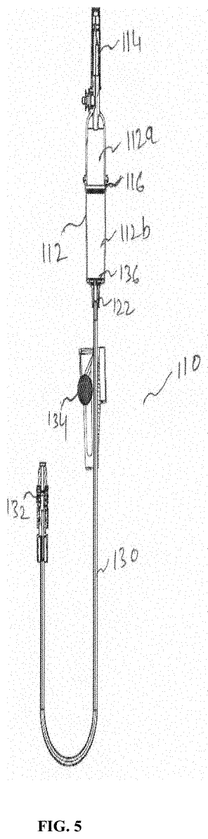

Referring to FIG. 5, an intravenous infusion set 110 according to another embodiment of the present invention is illustrated. The intravenous infusion set 110 comprises a drip chamber 112. The drip chamber 112 has two parts i.e. an upper part 112a and a lower part 112b, which are a hollow circular cylinder with a uniform wall thickness. Both the upper parts 112a and lower parts 112b can be flexible or rigid. Both of the parts 112a, 112b are connected by a joining member 116 in a leak proof arrangement. The flexible part of the drip chamber 112 is used for the purposes of priming. The upper part 112a of the drip chamber 112 is also provided with an air vent 118 with a cap. The air vent 118 has a filter and used for management of air in the infusion system. Alternatively, the drip chamber 112 can have a single structure.

The drip chamber 112 is provided with a spike 114 at a distal end of the upper part 112a. The spike 114 has a pointed tip which is used to pierce a fluid bag/bottle (not shown) so that the infusion fluid will be pulled by gravity into the drip chamber 112. The spike 114 is provided with slits or holes for fluid to flow into the drip chamber 112 from the fluid bag/bottle to the patient.

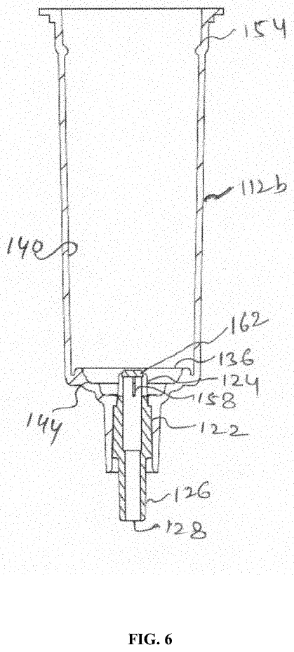

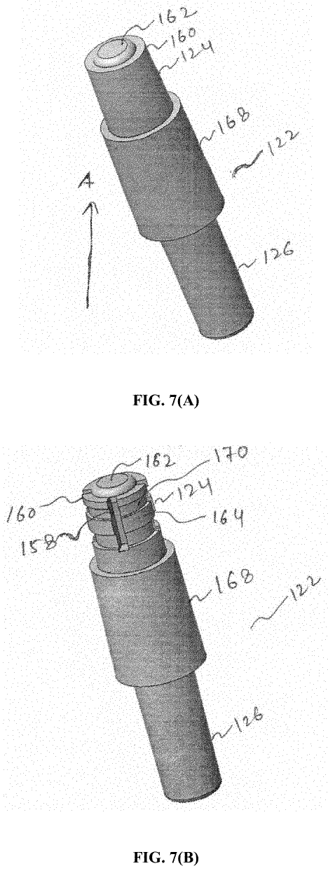

Referring now to FIG. 6 a lower part 112b of the drip chamber 112 is illustrated according to another embodiment of the present invention. The lower part 112b of the drip chamber 112 ends in an infusion fluid outlet 120 which is tubular and configured to house a flow path adapter 122. The flow path adapter 122 has an upper portion 124 having a distal extension 160, a middle portion 168 and a lower portion 126 and a through hole 128 therethrough for fluid flow. The dimension of the middle portion 168 has a bigger diameter than the dimension of the upper 124 and lower portion 126.

The lower portion 24 of the flow path adapter 122 is connected to a flexible infusion line 130 in a leak proof arrangement. The upper portion 124 of the flow path adapter 122 is received in the outlet 120 in a leak proof arrangement. One end of the flexible infusion line 130 of sufficient length as shown in FIG. 5 connects to a standard connector 132 at the patient end and other end of the infusion line 130 adjusted over the lower portion 126 of the flow path adapter 122 in a leak proof arrangement.

The proximal end of drip chamber 112 in particularly lower part thereof ends in an infusion fluid outlet 120 which is tubular and configured to house a flow path adapter 122. Said infusion fluid outlet 120 is covered with a fluid flow regulating member 136. The flow path adapter 122 is so adjusted and housed in the infusion fluid outlet 120 of the drip chamber 112 that the fluid flow regulating member 136 rests over the flow path adapter 122 impeccably once said fluid flow regulating member 136 is sealed by the means and methods such as energy director for ultrasonic welding or the like. The flow path adapter 122 is so housed that the gap between the fluid flow regulating member 136 and the distal extension 160 having a knob is minimal to ensure that the fluid passes through member 136 smoothly. Consideration is taken into account to take care for any damage to the fluid flow regulating member 136 due to interference between a distal end of flow path adapter 122 and the fluid flow regulating member 136 to avoid any functional failure.

The flow path adapter 122 has a unique design in that it eliminates collapsing of flexible infusion line 130 affecting flow of liquid. This unique design of the flow path adapter 122 also eliminates the chances of trapped air or bubbles going into the infusion line 130. The working principle behind such elimination of the trapped air or bubbles going into the infusion line 130 is that once the bubble or trapped air hits the upper part 124 having a distal extension 160 of the flow path adapter 122 i.e. one or more projections 164 and ridges/depression 170 provided therein, the bubble or trapped air breaks down. The area or passage provided is so less that any chances of trapped air or bubbles passing through one or more slits 158 provided in the flow path adapter 122 and into its passage 128 and in turn into the infusion line 130 is eliminated. Thus, the improved design of the flow path adapter 122 deals effectively with the most common yet serious issue of travelling of the bubble or trapped air into the infusion line 130 which is eliminated providing safety to the end user.

A fluid flow adjustment device, such as a roller clamp 134, is arranged being movable along the length of the flexible infusion line 130. The roller clamp 134 can be gradually adjusted so that the infusion line 130 could be squeezed proportionally to change the infusion rate and can also help in changing the fluid source, i.e. bottle or bag for multiple infusion procedures.

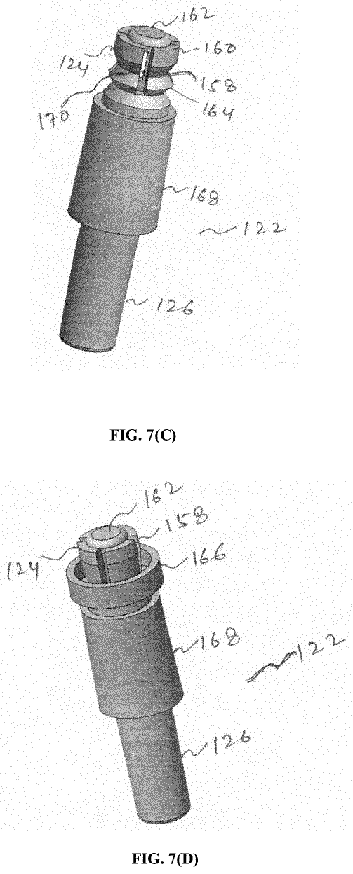

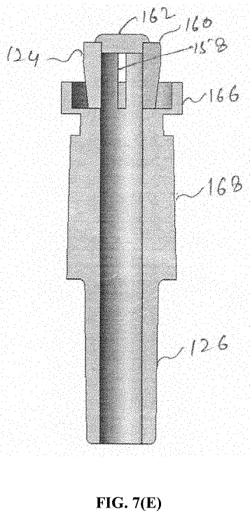

Referring to FIG. 7(A) to 7(D), a flow path adapter 122 according to one of the embodiments of the present invention is illustrated. The flow path adapter 122 has an upper portion 124 having a distal extension 160, a middle portion 168 and a lower portion 126 and a hole 128 therethrough for fluid flow. The lower portion 126 of the flow path adapter 122 is connected to a flexible infusion line 130 in a leak proof arrangement. The flow path adapter 122 in its upper portion 124 in particularly in the distal extension 160 comprises one or more slits 158. In the embodiment shown in FIG. 7(A), the flow path adapter 122 has at least one slit 158 (not shown). In the embodiments shown in FIGS. 7(B) to 7(E), the flow path adapter 122 has at least four slits 158, which are periodically placed across the distal extension 160 of the flow path adapter 122. The dimension of the slit(s) 158 is so designed so that it only allows the flow of fluid from the slits 158 and stop bubbles from entering into through hole 128 of the flow path adapter 122 and in turn into the infusion line 130. The through hole 128 i.e. opening at the top in the flow path adapter is sealed with a knob 162 in a leak proof arrangement. The distal extension 160 has one or more projections 164 and one or more ridges/depression 170 as shown in FIGS. 7(B) to 7(E) which work as a bubble buster and stop the flow of bubble into the infusion line 130.

The slits 158 are periodically placed in an axial direction on the distal extension 160 having a flow communication with the through hole 128 of flow path adapter 122. Alternatively, the slits 158 can be arranged in a direction opposite to the Axial direction or combination or in other fashion. The dimension of the slits 158 is so designed so that it only allows the flow of fluid from the slits 158 and stop bubble or trapped air to get into the through hole 128 and further into the infusion line 130. The slits can be designed in any shape and size.

The slits 158 are dimensioned such that air bubbles or trap do not pass through the slit 158. In another embodiment, the edge of the slits 158 may be serrated/rough to improve the desired bubble bursting or bubble breaking feature as part of a further improved bubble bursting mechanism.

Referring now to FIGS. 7B to 7C, the distal extension 160 of the flow path adapter 122 comprises a knob 160 and one or more projections 164 and one or more ridges/depression 170. The projections 164 and ridges 170 further work as bubble buster and the moment a bubble or air trap comes into contact with the edges projections 164 and ridges 170 it breaks. Thereby, the projections 164 and ridges 170 ensure that no bubble or air trap passes into the through hole 128 of the flow path adapter and further into the infusion line 130. The knob 160 completely blocks the opening of the through hole 128 and the fluid passing through the fluid flow regulating member 136 enters into the flow path adapter through the slits 158. The difference in shape and dimension of projections 164 and ridges 170 can be seen in FIGS. 7B and 7C. The shape and dimension of the projections 164 and ridges 170 may vary. In alternative embodiments, projections 164 and ridges 170 can have different shapes for example, spiral, curved, part-curved, or the like.

Referring now to FIG. 7D a perspective view of the flow path adapter 122 according to another embodiments of the present invention is illustrated. FIG. 7E is the cross-section of FIG. 7D. The flow path adapter 122 comprises one or more slits 158 for the flow of fluid. The distal extension 160 of the flow path adapter 122 at its proximal end has a circular fitment 166 which acts as a reservoir for the fluid to pass smoothly through the slits 158 and further into the through hole 128 and into the infusion line 130. The mechanism of fluid flow is already explained in the foregoing paragraphs.

Alternatively, or in addition to the slits, one or more holes, perforations, openings (not shown) or the combination thereof can be provided in the flow path adapter. Such holes, perforations, openings, improves the bubble bursting mechanism and ensure that any errant air bubble that may have entered through the fluid flow regulating member popped and does enter into the infusion line. The positions of such slits, perforations, opening can vary, for example in addition to keeping the slits in the distal extension such slit can be provided anywhere in between the proximal to distal end of the flow path adapter.

In a still further embodiment, the drip chamber 112 and its components are unitarily constructed and manufactured.

In yet another embodiment, the drip chamber 112 and its components are constructed and manufactured separately and assembled as one device.

In yet another embodiment, the lower portion of the drip chamber 112 can be made into two parts with the bubble bursting mechanism.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Thus, from the foregoing description, it will be apparent to one of ordinary skill in the art that many changes and modifications can be made thereto without departing from the scope of the invention as set forth in the claims.

Accordingly, it is not intended that the scope of the foregoing description be limited to the exact description set forth above, but rather that such description be construed as encompassing such features that reside in the present invention, including all the features and embodiments that would be treated as equivalents thereof by those skilled in the relevant art.

Thus, it is intended that the scope of the present invention herein disclosed should not be limited by the particular disclosed embodiments described above but should be determined only by a fair reading of the appended claims.

### LIST OF REFERENCE NUMERALS

10, 110 intravenous infusion set

12, 112 drip chamber

12a, 112a upper part

12b, 112b lower part

14, 114 spike

16, 116 joining member

18, 118 air vent

20, 120 infusion fluid outlet

22, 122 flow path adapter

24, 124 upper portion

26, 126 lower portion

28, 128 through hole

30, 130 flexible infusion line

32, 132 standard connector

34, 134 roller clamp

36, 136 fluid flow regulating member

38 sealing leg or sealing ring

40, 140 inner wall

42, 142 inner area

44, 142 circular rim

46 proximal face

48 distal face

50 outer area

52, 152 proximal end

54, 154 distal end

56 rim

158 slit

160 distal extension of the flow path adapter

162 knob

164 projections

166 fitment

168 middle portion

170 ridges/depression

A Axial direction

1. An intravenous infusion set (10) to administer delivery of intravenous fluid to a patient, said intravenous infusion set (10) comprising: a drip chamber (12), a flexible infusion line (30) of sufficient length for fluid delivery to the patient and connected the drip chamber (12); a roller clamp (34) arranged movably along the length of the flexible infusion line (30), an air vent (18) with a cap arranged at the distal end (54) of the drip chamber (12) for management of air in the infusion system, said drip chamber (12) has a spike (14) on a distal end (54) and an infusion fluid outlet (20) at a proximal end (52) of the drip chamber (12), a fluid flow regulating member (36) sealed within the drip chamber (12) over said infusion fluid outlet (20) and wherein said infusion fluid outlet (20) configured to house a flow path adapter (22) which facilitates in administering continuous air free delivery of intravenous fluid to patients under gravitational pull.

2. The improved intravenous infusion set (10) as claimed in claim 1, wherein the flow path adapter (22) comprises an upper portion (24), a lower portion (26) and a through hole (28) therethrough forming a passage for fluid flow.

3. The improved intravenous infusion set (10) as claimed in claim 2, wherein the lower portion (26) of the flow path adapter (22) connected to the flexible infusion line (30) in a leak proof of arrangement and the upper portion (24) of the flow path adapter (22) received in the infusion fluid outlet (20) in a leak proof arrangement.

4. The improved intravenous infusion set (10) as claimed in claim 1, wherein the drip chamber (12) comprising an upper part (12a) and a lower part (12b) connected by a joining member (16) in a leak proof arrangement.

5. The improved intravenous infusion set (10) as claimed in claim 1, wherein said fluid flow regulating member (36) is a hydrophilic membrane being permeable to fluid and impermeable to air.

6. The improved intravenous infusion set (10) as claimed in claim 1, wherein the fluid flow regulating member (36) sealed on a sealing leg or ring (38) which extends within the circumference of an inner wall (40) adjacent to the infusion fluid outlet (20) in the lower part (12b) of the drip chamber (12).

7. The improved intravenous infusion set (10) as claimed in claim 6, wherein the sealing leg or ring (38) circularly arranged being distanced from the inner wall (40) of the lower part (12b) of the drip chamber (12).

8. The improved intravenous infusion set (10) as claimed in claim 6, a circular rim (44) adjoining circumferentially the inner wall (42) provided below the sealing leg or ring (38) in an inner area (42).

9. The improved intravenous infusion set (10) as claimed in claim 6, wherein the fluid flow regulating member (36) has a proximal face (46) and a distal face (48) and the proximal face (46) of the member (36) includes a sealing area.

10. The improved intravenous infusion set (10) as claimed in any one of claims 1 to 9, wherein the flow regulating member (36) can be sealed to said sealing leg or ring (38) by heat sealing, adhesive sealing, ultrasonic welding, heated die, radio frequency, mechanical seal, insert molding, laser welding, press/snap fit, and annular ring with groove fitment.

11. An intravenous infusion set (110) to administer delivery of intravenous fluid to a patient comprising: a drip chamber (112), a flexible infusion line (130) of sufficient length for fluid delivery to the patient and connected to the drip chamber (112); a roller clamp (134) arranged movably along the length of the flexible infusion line (130), an air vent (118) with a cap arranged at the distal end (154) of the drip chamber (112) for management of air in the infusion system, said drip chamber (112) has a spike (114) on a distal end (154) and an infusion fluid outlet (120) at a proximal end (152) of the drip chamber (112), a fluid flow regulating member (136) sealed within the drip chamber (112) over said infusion fluid outlet (120) and wherein said infusion fluid outlet (120) configured to house a flow path adapter (122) which facilitates in administering continuous air free delivery of intravenous fluid without bubbles or air trap to patients under gravitational pull.

12. The intravenous infusion set (110) as claimed in claim 11, the flow path adapter (122) has an upper portion (124) having a distal extension (160), a middle portion (168) and a lower portion (126) and a through hole (128) therethrough for fluid flow.

13. The intravenous infusion set (110) as claimed in claim 11, the flow path adapter (122) so adjusted and housed in the infusion fluid outlet (120) of the drip chamber (112) that the fluid flow regulating member (136) rests over the flow path adapter (122).

14. The intravenous infusion set (110) as claimed in any preceding claim, the flow path adapter (122) in its upper portion (124) in particularly, in the distal extension (160) comprises one or more slits (158) periodically placed in an axial direction.

15. The intravenous infusion set (110) as claimed in claim 12, the through hole (128) i.e. opening at the top in the flow path adapter sealed with a knob (162) in a leak proof arrangement.

16. The intravenous infusion set (110) as claimed in claim 12, the distal extension (160) has one or more projections (164) and one or more ridges/depression (170).

17. The intravenous infusion set (110) as claimed in claim 16, the distal extension (160) of the flow path adapter (122) at its proximal end has a circular fitment (166) which acts as a reservoir for the fluid to pass smoothly through the slits (158) and further into the through hole (128) and into the infusion line (130).
